(19) United States
(12) Patent Application Publication
(10) Pub. No.: US 2025/0049698 A1
(43) Pub. Date: Feb. 13, 2025

(54) BIOACTIVE SERUM FRACTIONS FROM FRESH ROSE FLOWERS AND METHODS FOR THEIR PREPARATION AND USES

(71) Applicant: ISP INVESTMENTS LLC, Wilmington, DE (US)

(72) Inventors: Michael KOGANOV, White Plains, NY (US); Olga DUEVA-KOGANOV, White Plains, NY (US); Artyom DUEV, White Plains, NY (US); Li ZHANG, Princeton, NJ (US); Paul RECHT, Pleasantville, NY (US); Steven MICCERI, Milford, CT (US)

(73) Assignee: ISP INVESTMENTS LLC, Wilmington, DE (US)

(21) Appl. No.: 18/720,432

(22) PCT Filed: Dec. 14, 2022
(86) PCT No.: PCT/US22/52811

§ 371 Date: Jun. 14, 2024

Related U.S. Application Data
(60) Provisional application No. 63/290,786, filed on Dec. 17, 2021.

(51) Int. Cl. A61K 8/9789; A61Q 19/08

(52) U.S. Cl. CPC A61K 8/9789; A61Q 19/08; A61K 2800/51; A61K 2800/522; A61K 2800/524; A61K 2800/805

(57) ABSTRACT

The present invention relates to a method for obtaining a bioactive serum fraction derived from fresh rose flowers (Rosa spp.).

The present invention also provides bioactive serum fractions and bioactive extracts derived from fresh rose flowers (Rosa spp.) and compositions comprising the same.

The present invention further relates to a method of cosmetic treatment for improving skin appearance associated with antioxidant and antiaging properties of the bioactive serum fraction and the bioactive extract derived from fresh rose flowers (Rosa spp.).

### FIELD OF THE INVENTION

The present invention relates to the field of personal care. More particularly, it provides a method for obtaining bioactive serum fractions and bioactive extracts derived from fresh rose flowers (Rosa spp.).

The present disclosure also relates to bioactive serum fractions and bioactive extracts derived from fresh rose flowers (Rosa spp.).

Further the present invention relates to a method of cosmetic treatment for improving skin appearance associated with antioxidant and antiaging properties of the bioactive serum fraction and the bioactive extract derived from fresh rose flowers (Rosa spp.).

### BACKGROUND

Over the past several decades there has been a continuing need for higher quality botanical ingredients of enhanced purity and activity having fewer negative effects and which are solvent-free and prepared by environmentally friendly and sustainable methods. Current extraction methods fail to deliver the full spectrum of activities that exist within plant cells.

A rose is a woody perennial flowering plant of the genus Rosa, in the family Rosaceae, or the flower it bears. There are over three hundred species and tens of thousands of cultivars or hybrids.

In 2018, American Rose Society approved a new classification scheme that reflects both the botanical and evolutionary progress of the rose. There are three main groupings: Species (i.e., wild roses); Old Garden Roses (classes in existence before 1867); and Modern Roses (classes not in existence before 1867).

The genus Rosa is composed of 140-180 wild species and divided into four subgenera: Hulthemia, Hesperrhodos, Platyrhodon and Rosa. The subgenus Rosa is divided into 11 sections, among which one section is also called Rosa. Species (i.e. wild roses) are mentioned by their Latin name and include Rosa arabica, Rosa blanda, Rosa chinensis, Rosa gallica, Rosa glauca and Rosa californica (https://en.wikipedia.org/wiki/List_of_Rosa_species). There also exist thousands of hybrids among which hybrid Tea rose, Floribunda rose, Rosa X centifolia, Rosa x damascene or Rosa x alba and Rosa “Jardin de Granville”.

The rose has antioxidant effect and can help skin resist aging and maintain healthy and young look (Masek A. et al., Antioxidant properties of rose extract (Rosa villosa L.) measured using electrochemical and UV/Vis spectrophotometric methods. Int. J. Electrochem. Sci. 2017; 12:10994-11005); the effective components in the rose have strong moisturizing, water locking functions and whitening effect.

Many of the current extraction and separation methods yield crude botanical extracts that contain biological or chemical contaminants that can cause a loss of bioactivity potency, increased cytotoxicity, and decreased shelf life. Further, in order to yield a more refined botanical extract, current extraction methods often require the use of harsh chemical solvents.

Solvent-free extraction methods are also known from prior art. For example, a method using microwave to carry out water-free hydro-distillation and a method of extracting a volatile natural substance from a biological material are described in respectively patent U.S. Pat. No. 7,001,629 and patent application US 2004/0187340.

Furthermore, aqueous extracts of rose are disclosed in Chinese patents No. CN105249476 and CN103666766.

However, the rose skin care products currently available on the market are complex and miscellaneous, and the use of the product may result in skin allergy. Furthermore, some products are added with a low content of rose active ingredients and cannot play a certain skin care effect at all.

Therefore, there is a need to develop a composition truly containing natural rose extract to meet the consumer's demand for rose skin care products. Accordingly, there is a need for a method for preparing bioactive botanical compositions from plants that preserves the integrity of bioactive components and yields consistent results from lot-to-lot. Further, bioactive botanical compositions that could meet the industry standards with respect to shelf life, cytotoxicity, quality, and performance are needed in the cosmetic industry.

The solution to the technical problem addressed lies in the use of fresh rose flowers (Rosa spp.) serum fractions. The invention and the resulting advantages will be better understood upon reading the description.

### SUMMARY

The present disclosure relates to a method for obtaining a bioactive serum fraction derived from fresh rose flowers (Rosa spp.) comprising the steps of cleaning, macerating, pressing and mechanical separation of fresh rose flowers to obtain an intracellular colloidal dispersion (ICD) and a fiber enriched material (fraction A); treatment A and mechanical separation of the intracellular colloidal dispersion to obtain supernatant A (cytoplasm/cytosol fraction) and a membrane fraction; (fraction B); treatment B and mechanical separation of the supernatant A to obtain supernatant B (cytosol fraction) and fraction C (cytoplasm fraction); and treatment C and mechanical separation of the supernatant of supernatant B to yield the serum fraction and a fraction D (precipitate), wherein no exogenous solvent or liquid is added prior or during separating steps, as depicted in FIG. 1.

The present disclosure also relates to bioactive serum fractions and bioactive extracts derived from fresh rose flowers (Rosa spp.), obtainable by the method according to the invention, with the proviso that the Rosa “Jardin de Granville” is excluded. Further, the bioactive serum fractions and bioactive extracts have antioxidant and/or antiaging properties.

In another aspect the present disclosure also relates to a composition comprising an effective amount of the bioactive extract derived from fresh rose flowers (Rosa spp.) of the invention and a physiologically acceptable medium, with the proviso that the Rosa “Jardin de Granville” is excluded.

In a particular aspect the present disclosure relates to a composition intended for skin care topical application, with the proviso that the Rosa “Jardin de Granville” is excluded.

In another aspect the present disclosure relates to a method of cosmetic treatment for improving skin appearance associated with antioxidant and antiaging properties of the bioactive extract derived from fresh rose flowers (Rosa spp.), comprising applying to the skin the composition of the invention, with the proviso that the Rosa “Jardin de Granville” is excluded.

The present disclosure also relates to the use of the composition of the invention for skin care topical application, with the proviso that the Rosa “Jardin de Granville” is excluded.

The present disclosure also relates to various uses of the bioactive serum fraction and bioactive extract derived from fresh rose flowers (Rosa spp.) including functional food and functional beverages.

### BRIEF DESCRIPTION OF THE DRAWINGS

For the purpose of illustrating aspects of the present disclosure, certain embodiments of the invention are depicted in the drawing. However, the invention is not limited to the precise arrangements and instrumentalities of the embodiments depicted in the drawings.

FIG. 1. is a schematic drawing of the fractionating process for preparing the bioactive serum fraction and extract derived from fresh rose flowers

### DETAILED DESCRIPTION

Detailed embodiments of the present invention are disclosed herein; however, it is to be understood that the disclosed embodiments are merely illustrative of the invention that may be embodied in various forms. Therefore, specific structural and functional details disclosed herein are not to be interpreted as limiting, but merely as a representative basis for teaching one skilled in the art to variously employ the present invention.

### Definitions

Whenever a term is identified by reference to a range, the range will be understood to explicitly disclose every element thereof. As a non-limiting example, a range of 1-10% will be understood to include 1%, 2%, 3%, 4%, 5%, 6%, 7%, 8%, 9%, and 10%, and all values between 1 and 10%.

Where two or more substituents are referred to as being “selected from” a group of enumerated alternatives, it is meant that each substituent can be any element of that group, independent of the identity of the other substituents.

As used herein, “% refers to % by weight, that is the weight percent of a component in relation to the total weight of the skin care composition (i.e., including any carriers, vehicles, solvents, fillers, or other components added before application to the skin) unless otherwise provided.

All terms used herein are intended to have their ordinary meaning unless otherwise provided. For the purposes of describing and claiming the present invention, the following terms are defined:

The compositions described and used in the present disclosure can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

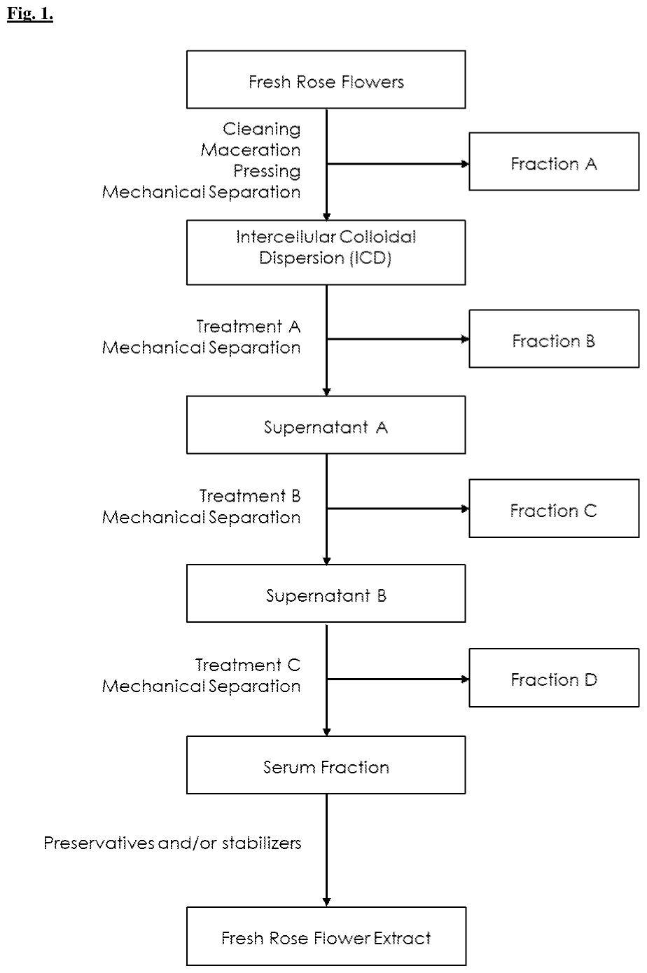

As used herein the terms “Serum Fraction” or “Bioactive Serum Fraction” mean a composition produced by a general method wherein no exogenous solvent or liquid is added prior or during said method, comprising the main steps of: of cleaning, macerating, pressing and mechanical separation of fresh rose flowers (Rosa spp.) to obtain an intracellular colloidal dispersion (ICD) and a fiber enriched material (fraction A); treatment A and mechanical separation of the intracellular colloidal dispersion to obtain supernatant A (cytoplasm/cytosol fraction) and a membrane fraction; (fraction B); treatment B and mechanical separation of the supernatant A to obtain supernatant B (cytosol fraction) and fraction C (cytoplasm fraction); and treatment C and mechanical separation of the supernatant of supernatant B to yield the serum fraction and a fraction D (precipitate), to yield the Serum Fraction and a Fraction C (precipitate), as illustrated in FIG. 1, and disclosed in EP2919757, JP 6130924, CN ZL201380057567.6, EP2491939B1, CN1929851B, U.S. Pat. Nos. 8,734,861 and 7,473,435; U.S. patent application Ser. No. 16/078,925, incorporated herein for reference.

“Extract” or “Bioactive extract” or “Rose flower extract” as used herein means a combination of a serum fraction of fresh rose flowers and preservatives and/or stabilizers to protect composition of the ingredient against expected environmental challenges such as temperature, atmosphere (e.g., oxygen), light, and microorganisms, as illustrated in FIG. 1

“Fresh rose flowers” or “rose flowers” as used herein means live flowers of rose (Rosa spp.) including petals or including petals, pistil and stamen, e.g., flowers harvested and stored at 4° C. until sufficient biomass is collected with total exposure less than 8 hours before preparing a Serum Fraction or an Extract.

“Rosa spp.” as used herein has the meaning of any species of the Genus Rosa, subgenus Rosa.

“Rosa Freedom” is the name of a large flowering hybrid tea rose.

Rosa “Jardin de Granville” is a modern hybrid variety created by «Roses anciennes André Eve S.A.S» at the request of the group Parfums Christian Dior and protected by the French Plant Variety Certificate No 20110345 (name Rosa L., variety EVANRAT).

“Effective amount” as used herein means an amount of a compound or skin care composition sufficient to significantly induce a positive appearance and/or feel benefit, but low enough to avoid serious side effects (i.e., to provide a reasonable benefit to risk ratio, within the scope of sound judgment of the skilled artisan).

“Cleaning” refers to removal of debris from fresh rose prior to further processing, in a way that avoids injury to the plant, or removal of valuable components. For example, it can be performed by low-pressure rinsing with potable water under conditions where runoff water wash would not noticeably contain plant pigments. Excess wash water is then removed from the washed plants.

“Maceration” refers to rendering fresh rose into smaller particles to disrupt its integrity and ease the following expelling of liquid intracellular colloidal dispersion. Examples of suitable maceration implements include, but are not limited to, devices such as a crusher, a grinder, or a mill (e.g., knife mill, hammer mill, etc.). To prevent temperature-induced degradation of plant material, maceration step can include temperature monitoring and selection of maceration parameters ensuring that there is no significant rise in temperature of plant material during this step.

“Pressing” refers to separating liquid material from fresh rose by application of mechanical force. This includes, but is not limited to, techniques such as draining by ambient gravity, pressing by a heavy object, centrifugal force from a rotary expeller, pressure from piston of a hydraulic press, or rollers or a screw of appropriate type of press.

“Fiber enriched material” (FEM) refers to fiber-enriched solid and/or semi-solid fraction of fresh rose from which the liquid intracellular dispersion has been removed by pressing.

“Intracellular Colloidal Dispersion” (ICD) refers to liquid material expelled by pressing fresh rose. Resulting liquid contains dispersed solid and/or semi-solid particles and possible droplets of water-immiscible liquids of a variety of sizes (collectively referred to as particles), in contiguous aqueous medium. The particles are mainly comprised of plant cell organelles, organelle fragments, and residual fiber-enriched material. The aqueous medium is mainly comprised of cytosol and vacuole contents.

“Adjustment” refers to alteration of activities of hydroxide and hydronium ions in aqueous medium of intracellular dispersion or aqueous fractions produced by further processing of intracellular dispersion, with activity of hydronium ion remaining within range found in viable plant cells (e.g., between pH 3 and pH 9). This alteration can be accomplished by, for example, an electro-membrane process (e.g., being passed through an electrodialysis chamber with bipolar membranes), or by addition of an acid or an alkali. Adjustment parameters are selected to be sufficient for a particular change in physico-chemical parameters, such as pH, or work function value, or surface potential at electrolyte-air interface. Such adjustments facilitate following destabilization and/or separation steps; or create conditions for proper preservation and stabilization.

“Destabilization” refers to treating adjusted ICD using electromagnetic waves for transiently modifying physical properties (such as ε₀′ which is the real component of low-frequency dielectric constant). This treatment degrades the stability of the ICD by causing agglomeration and/or aggregation of particles into assemblies which are sufficiently large and stable to enable and/or improve following separation into fractions with certain desirable properties.

The terms “Separation” or “Mechanical separation” refer to separating solid and/or semisolid particles and non-aqueous liquid droplets from aqueous liquid by exploiting density and/or size of particles. This includes but is not limited to techniques such as straining, filtration (including filtration utilizing a pressure gradient), skimming, sedimentation by ambient gravity, decanting, centrifugation, or some combination of the above. Continuous flow mechanical separation has been used, but this does not exclude batch processing.

“Supernatant” refers to aqueous material from which particles have been separated. “Supernatant A” and “supernatant B” refer to supernatants resulting from respective separation steps of the process.

“Precipitate” refers to particles from which aqueous material has been separated. “Fraction B” and “fraction C” refer to precipitates resulting from respective separation steps of the process.

“Preservatives and/or stabilizers” refers to substances which, when added to a serum fraction of fresh rose, protect it against expected environmental challenges such as temperature, atmosphere (e.g., oxygen), light, and microorganisms. Particularly suitable substances can include, without limitation, a preservative, a stabilizer and/or mixture thereof.

“Apply” or “Application” as used in reference to a skin care composition means to apply or spread the compositions of the present invention onto a human skin surface such as the epidermis.

“Physiologically acceptable” as used herein means any compound adapted to come into contact with the skin or a mucous membrane without causing reactions of toxicity, intolerance, allergic response and the like.

As used herein, the term “Physiologically acceptable” in functional food and beverage products” refers to bioactive serum fractions, formulations or inert ingredients that are suitable for internal use without undue toxicity, incompatibility, instability, irritation, allergic response, and the like, commensurate with a reasonable benefit/risk ratio.

“Topical” refers to a composition that is intended to be applied to a bodily surface such as skin or hair.

As used herein, the term “topical application” generally refers to techniques relating to directly laying on or spreading formulations containing bioactive or bioactive extract onto the outer skin using, e.g., by use of the hands or an applicator such as a wipe.

“Functional food” or “functional beverages” as used herein mean food or beverages comprising ingredients that offer health benefits that extend beyond their nutritional value.

It is obvious that the invention concerns mammals in general, and more specifically human beings.

Method for Preparing a Serum Fraction and an Extract Derived from Fresh Rose Flowers

The present invention provides a method for obtaining a bioactive serum fraction derived from fresh rose flowers (Rosa spp.) comprising the steps of cleaning, macerating, pressing and mechanical separation of fresh rose flowers to obtain an intracellular colloidal dispersion (ICD) and a fiber enriched material (fraction A); treatment A and mechanical separation of the intracellular colloidal dispersion to obtain supernatant A (cytoplasm/cytosol fraction) and a membrane fraction; (fraction B); treatment B and mechanical separation of the supernatant A to obtain supernatant B (cytosol fraction) and fraction C (cytoplasm fraction); and treatment C and mechanical separation of the supernatant of supernatant B to yield the serum fraction and a fraction D (precipitate), wherein no exogenous solvent or liquid is added prior or during separating steps.

Reference is made to FIG. 1, which is a schematic of one embodiment of a method for processing fresh rose flowers including petals, pistil and stamen to produce a bioactive fraction in accordance with the present invention.

The present invention also allows for the standardization of initial plant material properties to improve reproducibility of bioactive (fractions) ingredients by exploring uniform conditions for rose (Rosa spp.) processing.

In an embodiment fresh rose flowers belong to any species of roses (wild roses), Old Garden Roses and Modern Roses (hybrids, varieties or cultivar), in reference to the American Rose Society classification.

In another embodiment fresh rose flowers are selected among Modern Rose including hybrids, varieties or cultivars, more preferably from fresh hybrid tea rose variety and even more preferably from fresh hybrid tea roses named Rosa Freedom.

In one embodiment of the process of the present invention, fresh live rose flowers including the petals, pistil and stamen were removed from the rose stems, including the sepal and receptacle. The harvesting was conducted in such a manner to avoid chopping or crushing of the collected biomass to avoid disruption of the flowers cell structure.

In one embodiment of present invention, collected flowers were spray rinsed with 10° C. to 15° C. water for 0.1 to 0.3 minutes at a rate of 5 to 6 liters per minute just prior to processing. Excess water was removed from the rinsed flowers by allowing to drain for at least 1 minute. The rinsed flowers then underwent maceration, pressing and separation utilizing a mechanical screw press (Model CP-6 Vincent Corporation, FL) to extract the liquid intercellular colloidal dispersion (ICD) content from the fiber enriched material (fraction A). The yield of fraction A was between 30% and 50% (w/w) and the intercellular colloidal dispersion contained from 5% to 12% dry matter.

In one embodiment of present invention, the ICD can be frozen at −20° C. for storage without harm for later use.

In one embodiment of present invention, treatment A is achieved by treatment of the ICD with electromagnetic waves produced from magnetrons operating at a frequency of between 2.45 and 5.8 GHz to decrease the value of real component of low-frequency dielectric constant (80) of the ICD by about 30 Farads per meter (from about 80 F/m to about 50 F/m) compared to its value prior to treatment.

In another particular embodiment, treatment A is achieved by treatment of the ICD with electromagnetic waves produced from magnetrons operating at a frequency of 2.45 GHz.

In another particular embodiment, treatment A is achieved by treatment of the ICD with electromagnetic waves produced from magnetrons operating at a frequency of 5.8 GHZ, which allows to reach same results while using less energy.

It was found that these modifications degrade the stability of intracellular dispersion causing agglomeration and/or aggregation of particles (i.e., organelles, organelle fragments, residual fibrous material) into assemblies which are sufficiently large and stable to enable and/or improve mechanical separation to supernatant A and fraction B.

The value of real component of low-frequency dielectric constant (ε₀′) was determined using broadband dielectric spectroscopy data obtained via equipment and software from Agilent Technologies: PNA-L Network Analyzer N5230C with 85070E dielectric probe kit, N4693-60001 electronic calibration module, and 85070 software. The calculation was performed according to method described in the article Cole, K. S., & Cole, R. H. (1941). Dispersion and absorption in dielectrics I. Alternating current characteristics. The Journal of Chemical Physics, 9(4), 341-351.

In another embodiment of present invention, treatment A with electromagnetic waves is preceded by decrease of pH level in ICD by titration with, for example, acid to obtain a pH lower than 4, preferably a pH ranging from 3 to 4, and more preferably to obtain a pH of 3.5.

Alternatively, treatment A with electromagnetic waves can be preceded by a filtration.

Mechanical separation of the ICD after treatment A can be achieved by using techniques such as straining, filtration (including utilizing pressure gradient), skimming, sedimentation under ambient gravity, decanting and centrifugation or combinations thereof.

In one embodiment of present invention, mechanical separation of the ICD after treatment A is achieved by centrifugation at 18,000 g for 45 minutes to produce supernatant A and fraction B.

In one embodiment of present invention, supernatant A has turbidity below about 100 NTU. Fraction B contains about 15.0% to 25.0% dry matter.

In one embodiment of present invention, supernatant A has osmolality of between 450 to 500 mOsm/kg H2O.

In one embodiment of present invention, treatment B is achieved by increase of pH level in supernatant A by titration with for example alkali, to obtain a pH ranging from 6.0 to 9.0.

In a particular embodiment of present invention, treatment B is achieved by increase of pH level in supernatant A by titration with for example alkali, obtain a pH greater than 6.0, more preferably a pH ranging from 6.5 to 7.5, and even more preferably to obtain a pH of 7.0.

Alternatively, treatment B can be achieved by increase of pH level in supernatant A by titration with for example alkali, to obtain a pH greater than 8.0, preferably a pH ranging from 8.5 to 9.0, even more preferably to obtain a pH of 9.0.

Mechanical separation of supernatant A after treatment B is achieved by using techniques such as straining, filtration (including utilizing pressure gradient), skimming, sedimentation under ambient gravity, decanting and centrifugation or combinations thereof.

In one embodiment of present invention, mechanical separation of the supernatant A after treatment B is achieved by centrifugation at 18,000 g for 45 minutes to produce supernatant B and fraction C.

In one embodiment of present invention, fraction C contains about 10.0% to 20.0% dry matter.

In one embodiment of present invention, destabilization treatment C is achieved by decrease of pH level in supernatant B by titration with, for example, acid to obtain a pH lower than 5, preferably a pH ranging from 3.5 to 4.5, for example to obtain a pH of 4.0.

Mechanical separation of supernatant B after treatment C is achieved by using techniques such as straining, filtration (including utilizing pressure gradient), skimming, sedimentation under ambient gravity, decanting and centrifugation or combinations thereof.

In one embodiment of present invention, mechanical separation of supernatant B after treatment C is achieved by centrifugation at 18,000 g for 45 minutes to produce serum fraction and fraction D.

In one embodiment of present invention serum fraction contains from 5.0% to 10.0% dry matter.

In one embodiment of present invention serum fraction has an osmolality of between 460 and 600 mOsm/kg H2O.

In one embodiment of present invention serum fraction has a dry matter between 5.0% and 10.0%.

The obtained serum fraction can be further processed by adding preservatives and/or stabilizer to prepare a rose flower extract.

Preservatives and stabilizers, when added to a serum fraction of fresh rose, protect it against expected environmental challenges such as temperature, atmosphere (e.g., oxygen), light, and microorganisms.

In one embodiment, suitable preservatives for use in the present invention include, for example, potassium sorbate and sodium benzoate.

In one embodiment, stabilizers include at least one chelating agent, at least one antioxidant, and at least one preservative efficacy booster.

In one embodiment, suitable stabilizers for use in the present invention include, for example, sodium phytate as chelating agent, ascorbic acid as antioxidant, and pentylene glycol as preservative efficacy booster.

In one embodiment the preservative and stabilizer mixture represent 0.2% to 0.75% of the rose flower extract.

This does not exclude possibility of a serum fraction free of preservatives and stabilizers being created by using particular processing and packaging techniques.

The isolated finished serum fraction or extract can be further concentrated and then stabilized for further utilization in skin care for topical application, functional food and beverages applications.

The bioactive serum fraction or extract of the present invention can further be included in delivery systems that are commonly used in the art.

In another aspect the present disclosure also relates to fraction A (fiber enriched material), fraction B (membrane fraction); fraction C (cytoplasm fraction) and fraction D (precipitate) that can be used for pharmaceutical, nutraceutical, flavor and fragrance applications.

Bioactive Serum Fraction and Extract Derived from Fresh Rose Flowers

The present invention relates to a bioactive serum fraction derived from fresh rose flowers (Rosa spp.), with the proviso that the Rosa “Jardin de Granville” is excluded.

In an embodiment the bioactive serum fraction is derived from fresh rose belonging to any species of roses (wild roses), Old Garden Roses and Modern Roses (hybrids, varieties or cultivar), in reference to the American Rose Society classification.

In another embodiment the bioactive serum fraction is derived from fresh Modern Rose including hybrids, varieties or cultivars.

In a particular embodiment the bioactive serum fraction is derived from fresh hybrid tea rose variety.

In another particular embodiment the bioactive serum fraction is derived from fresh hybrid tea rose Rosa Freedom.

In a particular embodiment the bioactive serum fraction derived from fresh rose flowers (Rosa spp.) is obtained by the method for preparing a serum fraction derived from fresh rose flowers described above.

In one embodiment of present invention serum fraction contains from 5.0% to 10.0% dry matter.

In another embodiment the bioactive serum fraction obtained by the above method is mixed with preservatives and/or stabilizers to provide a bioactive extract.

### Composition

In another aspect the present disclosure also relates to a composition comprising a physiologically acceptable medium and an effective amount of a bioactive extract derived from fresh rose flowers (Rosa spp.) obtainable by the method according to the invention, with the proviso that the Rosa “Jardin de Granville” is excluded.

In one embodiment, the rose flowers are chosen from rose belonging to any species of roses (wild roses), Old Garden Roses and Modern Roses, preferably from Modern Rose including hybrids, varieties or cultivars, more preferably from hybrid tea rose variety and even more preferably from the hybrid tea rose Rosa Freedom, with the proviso that the Rosa “Jardin de Granville” is excluded.

In one embodiment the composition is intended for topical application and is selected from the group consisting of an aqueous, hydro-alcoholic or oily solution; and oil-in-water emulsion, a water-in-oil emulsion or multiple emulsions; a suspension or a powder.

In one embodiment the topical skin care composition is in the form of a leave-on product selected from the group consisting of a cream, a dressing, a gel, a lotion, an ointment, a liquid, a spray applicator, and combinations thereof, or a wash-off product selected from the group consisting of hand dishwashing detergent, liquid hand soap, bar soap, body wash, shampoo, general purpose cleanser, and combinations thereof.

In another aspect the present disclosure relates to a method of cosmetic treatment for improving skin appearance associated with antioxidant and antiaging properties of the bioactive serum fraction and the bioactive extract derived from fresh rose flowers (Rosa spp.), comprising applying to the area to be treated a skin care composition comprising a physiologically acceptable medium and an effective amount of a bioactive extract derived from fresh rose flowers (Rosa spp.) obtainable by the method according to the invention, wherein the rose flowers are chosen from rose belonging to any species of roses (wild roses), Old Garden Roses and Modern Roses, preferably from Modern Rose including hybrids, varieties or cultivars, more preferably from hybrid tea rose variety and even more preferably from the hybrid tea rose Rosa Freedom, with the proviso that the Rosa “Jardin de Granville” is excluded.

In another aspect the present disclosure relates to the use of the composition according to the invention, i.e. obtainable by the method according to the invention wherein the rose flowers are chosen from rose belonging to any species of roses (wild roses), Old Garden Roses and Modern Roses, preferably from Modern Rose including hybrids, varieties or cultivars, more preferably from hybrid tea rose variety and even more preferably from the hybrid tea rose Rosa Freedom, with the proviso that the Rosa “Jardin de Granville” is excluded, for skin care topical application.

In another aspect the present disclosure relates to the use of the composition according to the invention, i.e. obtainable by the method according to the invention wherein the rose flowers are chosen from rose belonging to any species of roses (wild roses), Old Garden Roses and Modern Roses, preferably from Modern Rose including hybrids, varieties or cultivars, more preferably from hybrid tea rose variety and even more preferably from the hybrid tea rose Rosa Freedom, with the proviso that the Rosa “Jardin de Granville” is excluded for functional food or functional beverages.

### EXAMPLES

The present invention is now described with reference to the following examples. These examples are provided for the purpose of illustration only and the invention should in no way be construed as being limited to these examples, but rather should be construed to encompass any and all variations which become evident as a result of the teaching provided herein. Thus, the following examples are intended to illustrate particular embodiments of the present invention but are by no means intended to limit the scope of the present invention.

### Example 1 Preparation of Bioactive Serum Fraction and Extract #2012 Derived from Rosa Freedom Flower

Fresh live rose flowers including the petals, pistil and stamen were removed from the Rosa Freedom stems including the sepal and receptacle and 4-5 cm of stem were harvested from rose plants. The harvesting was conducted in such a manner to avoid chopping or crushing of the collected biomass to avoid disruption of the flowers cell structure. Viability of the collected plants was tested utilizing an OS5p multi-mode chlorophyll fluorometer (Opti-Sciences Inc, Hudson, NH, USA). The fresh live flowers including the petals, pistil and stamen were removed from the stem including the sepal and receptacle.

The steps of cleaning, macerating, pressing and separating are conducted as described above in the section “Method for Preparing a Serum Fraction and an Extract Derived from Fresh Rose Flowers” and as illustrated in FIG. 1.

The obtained ICD was frozen at −20° C. for storage for at least 16 hours.

A representative sample of frozen Rosa Freedom flower ICD was gently thawed in water bath at 25° C. The thawed ICD then underwent further destabilization treatments as per FIG. 1. treatment A was achieved by filtration and subsequent treatment by electromagnetic waves operating at a frequency of 2.45 GHz in a continuous flow system that includes magnetrons. The said electromagnetic waves decreased the value of real component of low-frequency dielectric constant (ε₀′) of intracellular dispersion during the treatment by about 30 Farads per meter (F/m)—compared to its value prior to treatment (i.e., from 80 F/m to 50 F/m). The electromagnetic treated ICD was then mechanically separated by centrifugation at 18,000 g for 45 minutes to produce fraction B and supernatant A. Treatment B was then achieved by increase of pH of supernatant A by titration with alkali to about pH=7.0. The pH adjusted supernatant A was then separated by centrifugation at 18,000 g for 45 minutes to produce fraction C and supernatant B. Treatment C was achieved by decrease of pH level in supernatant B by titration with acid to about pH=4.0. The pH adjusted supernatant B was then separated by centrifugation at 18,000 g for 45 minutes to produce fraction D and serum fraction. Preservatives and/or stabilizers were then added to serum fraction to produce rose flower extract #2012.

### Example 2 Preparation of Bioactive Serum Fraction and Extract #2013 Derived from Rosa Freedom Flower

A representative sample of frozen Rosa Freedom flower ICD as described in Example 1 was gently thawed in water bath at 25° C. The thawed ICD then underwent further destabilization treatments as per FIG. 1. Treatment A was achieved by filtration and subsequent treatment by electromagnetic waves operating at a frequency of 2.45 GHz. in a continuous flow system that includes magnetrons. The said electromagnetic waves decreased the value of real component of low-frequency dielectric constant (ε₀′) of intracellular dispersion during the treatment by about 30 Farads per meter (F/m)—compared to its value prior to treatment (i.e., from 80 F/m to 50 F/m). The electromagnetic treated ICD was then mechanically separated by centrifugation at 18,000 g for 45 minutes to produce fraction B and supernatant A. Treatment B was then achieved by increase of pH of supernatant A by titration with alkali to about pH=9.0. The pH adjusted supernatant A was then separated by centrifugation at 18,000 g for 45 minutes to produce fraction C and supernatant B. Treatment C was achieved by decrease of pH level in supernatant B by titration with acid to about pH=4.0. The pH adjusted supernatant B was then separated by centrifugation at 18,000 g for 45 minutes to produce fraction D and serum fraction. Preservatives and/or stabilizers were then added to serum fraction to produce rose flower extract #2013.

### Example 3 Preparation of Bioactive Serum Fraction and Extract #2014 Derived from Rosa Freedom Flower

A representative sample of frozen Rosa Freedom flower ICD as described in Example 1 was gently thawed in water bath at 25° C. The thawed ICD then underwent further destabilization treatments as per FIG. 1. Treatment A was achieved by decrease of pH level in ICD by titration with acid to about pH=3.5. The pH adjusted ICD was then subsequently treated by electromagnetic waves operating at a frequency of 2.45 GHz in a continuous flow system that includes magnetrons. The said electromagnetic waves decreased the value of real component of low-frequency dielectric constant (ε₀′) of intracellular dispersion during the treatment by about 30 Farads per meter (F/m)—compared to its value prior to treatment (i.e., from 80 F/m to 50 F/m). The electromagnetic treated ICD was then mechanically separated by centrifugation at 18,000 g for 45 minutes to produce fraction B and supernatant A. Treatment B was then achieved by increase of pH of supernatant A by titration with alkali to about pH=7.0. The pH adjusted supernatant A was then separated by centrifugation at 18,000 g for 45 minutes to produce fraction C and supernatant B. Treatment C was achieved by decrease of pH level in supernatant B by titration with acid to about pH=4.0. The pH adjusted supernatant B was then separated by centrifugation at 18,000 g for 45 minutes to produce fraction D and serum fraction preservatives and/or stabilizers were then added to serum fraction to produce rose flower extract #2014.

### Example 4 Preparation of Bioactive Serum Fraction and Extract #2015 Derived from Rosa Freedom Flower

A representative sample of frozen Rosa Freedom flower ICD as described in Example 1 was gently thawed in water bath at 25° C. The thawed ICD then underwent further destabilization treatments as per FIG. 1. Treatment A was achieved by decrease of pH level in ICD by titration with acid to about pH=3.5. The pH adjusted ICD was then subsequently treated by electromagnetic waves operating at a frequency of 2.45 GHz in a continuous flow system that includes magnetrons. The said electromagnetic waves decreased the value of real component of low-frequency dielectric constant (ε₀′) of intracellular dispersion during the treatment by about 30 Farads per meter (F/m)—compared to its value prior to treatment (i.e. from 80 F/m to 50 F/m). The electromagnetic treated ICD was then mechanically separated by centrifugation at 18,000 g for 45 minutes to produce fraction B and supernatant A. Treatment B was then achieved by increase of pH of supernatant A by titration with alkali to about pH=9.0. The pH adjusted supernatant A was then separated by centrifugation at 18,000 g for 45 minutes to produce fraction C and supernatant B. Treatment C was achieved by decrease of pH level in supernatant B by titration with acid to about pH=4.0. The pH adjusted supernatant B was then separated by centrifugation at 18,000 g for 45 minutes to produce fraction D and serum fraction. Preservatives and/or stabilizers were then added to serum fraction to produce rose flower extract #2015.

### Example 5 Comparison of Biological Activities of Rosa Freedom Flower Extracts from Examples 1 to 4 for Selecting a Processing Regime Resulting in the Best Activities

### DPPH Free Radical Scavenging Assay

DPPH assay provides an easy and rapid way to evaluate potential antioxidants. A free radical is an atom or molecule that has one or more unpaired electrons. The evidence is that reactive species of oxygen (O2-, H2O2, and NO2+) play an important role in inflammation, neurodegenerative disorders, and cancer, etc. Antioxidants can react with the nitrogen centered radical DPPH (2,2-Diphenyl-1-Picrylhydrazyl) and convert it to the non-radical 2,2-Diphenyl-1-Picrylhydrazine. The changes in color (from deep violet to light yellow) were read at 517 nm after 60 min of reaction using a UV/VIS Spectrophotometer. DPPH free radical scavenging method offers the first approach for evaluating the antioxidant potential of a compound or extract.

Human skin is constantly directly exposed to the air, solar radiation, environmental pollutants, or other mechanical and chemical insults, which are capable of inducing the generation of free radicals and reactive oxygen species (ROS) that result in inflammation and extrinsic skin aging [Borut Poljšak and Raja Dahmane, Free Radicals and Extrinsic Skin Aging. Dermatol Res Pract. 2012:135206, 2012]. Free radical is a molecule or an atom with one or more unpaired valence shell electrons. Such substances are often but not always unstable, chemically transient and highly reactive. Free radicals can be produced by many processes including combustion, irradiation by sunlight, and normal metabolism—especially involving cellular respiration, immune response and inflammation processes. In biological systems, free radicals most commonly involve oxygen metabolism and reactive oxygen species. High reactivity of free radicals can let them damage biological molecules. In cases where products of such reactions are free radicals themselves, this can lead to a cascade of damage. Free radicals are both the earliest inflammation messengers and key parts of inflammatory damage mechanisms. Excess of free radicals contributes to self-sustaining loops of harmful inflammation. This is particularly relevant in the human skin as the organ most exposed to environmental stresses which generate free radicals. Quenching and scavenging these free radicals could help mitigate and prevent signs of skin damage and skin aging.

DPPH free radical scavenging activity was determined to assess antioxidant activities for test articles by following protocol. DPPH (Sigma-Aldrich, 300267, St. Louis, MO, USA) solution of 0.06 mM in absolute methanol was prepared. Test articles with different concentrations (20 μl) were mixed with 180 μl of DPPH solution to obtain a total of 200 μl per well in a 96-well plate. After 60 min incubation at room temperature in the dark, the absorbance was measured at 517 nm by using a microplate spectrophotometer Biotek Synergy 2 (Biotek Instruments Inc, Winooski, VT, USA). Each measurement was corrected with its background, which was the sample without DPPH solution. L-(+)-ascorbic acid (Vitamin C, Sigma-Aldrich, A5960, St. Louis, MO, USA) in 5% solution (w/v) as a standard was diluted as the same as the test articles. The ability to scavenge DPPH radicle was calculated as % inhibition. The antioxidant activities of all test articles were expressed as an IC50 value that is defined as the concentration of the test article showing 50% inhibition.

### Human Neutrophil Elastase Inhibition Assay

Elastin is essential in connective tissues which depend on elasticity for their function, such as skin. Excessive elastase activity, commonly related to inflammation, degrades elastin in elastic fiber network and decreases strength and resilience of the skin. Elastin is one of the dermal extracellular matrix components, which is responsible for maintaining the elasticity and resilience of the skin [Oikarinen A. Connective tissue and aging. Int. J. Cosmet. Sci. 26:107-8, 2004]. It is an insoluble elastic fiber protein along with collagen in influencing the mechanical properties of connective tissue [Antonicelli F, Bellon G, Debelle L, Hornebeck W. Elastin-elastases and inflamm-aging. Curr Top Dev Biol. 79:99-155, 2007]. During aging, the elasticity of the skin is reduced by the enzyme elastase which degrades elastin fibers and leads to sagging. Elastase belongs to the family of chymotrypsin, an enzyme that is capable of hydrolyzing materials such as elastin and fibrillin. The secretion and activation of elastase from dermal fibroblasts in response to sun radiation and/or to inflammatory cytokines released by keratinocytes are responsible for the degeneration of the three-dimensional structure of elastic fibers during the formation of wrinkles in skin aging process.

Human neutrophil elastase enzymatic activity was determined with the EnzChek™ Protease Assay Kit (E6638) according to manufacturer's instruction (Molecular Probes, Inc. Eugene, OR, USA). Human neutrophil elastase prepared from neutrophils was used (Sigma-Aldrich, 324681-50UG, 1 unit, St. Louis, MO, USA). Test articles and control (20 μl) were preincubated with the enzyme (90 μl) at final concentration of 280 ng/ml in 10 mM Tris-HCl buffer (pH 7.8) for 15 min at room temperature. The reaction was initiated by adding BODIPY FL casein substrate (90 μl) and was followed by incubation in the dark for 1 h at room temperature. A selective elastase inhibitor, N-(MeOsuc)-Ala-Ala-Pro-Val-chloromethyl ketone (Sigma-Aldrich, M0398-5 MG, St. Louis, MO, USA), was used as a positive control. Fluorescence intensity was measured at excitation/emission of 485/530 nm by using a microplate spectrophotometer Biotek Synergy 2 (Biotek Instruments Inc, Winooski, VT, USA). All values were corrected for background fluorescence. The elastase inhibitory activities of all test articles were expressed as an IC50 value that is defined as the concentration of the test article showing 50% inhibition.

### Results

Four rose flower extracts (#2012, #2013, #2014, and #2015) obtained in Examples 1 to 4 were tested in both DPPH free radical scavenging assay and elastase inhibition assay. The results are shown in Table 1.

As shown in Table 1, both DPPH free radical scavenging and elastase inhibition activities of all test articles were expressed as IC50. The lower IC50 value indicates the more desired activities, thus positive antioxidant, anti-inflammatory and antiaging benefits. The rank of rose flower extracts for DPPH free radical scavenging activities from high to low was the following: #2012 (0.16%)>#2014 (0.20%)>#2013 (0.26%)>#2015 (0.30%). Rosa Freedom flower extract #2012 demonstrated IC50 at 0.16, which was the highest antioxidant activity among the four rose flower extracts in Examples 1 to 4. A positive control, L-(+)-ascorbic acid (Vitamin C), indicated IC50 at 0.01% (v/v) of a dilution from 5% (w/v) stock solution. In addition, the rank of rose flower extracts for inhibiting human neutrophil elastase activities from high to low was the following: #2012 (0.00002%)>#2014 (0.00003%)>#2013 (0.001%)>#2015 (0.005%). Rosa Freedom flower extract #2012 indicated IC50 at 0.00002%, which was the highest elastase inhibition activity among the four rose flower extracts in Examples 1 to 4. A selective elastase inhibitor, N-(MeOsuc)-Ala-Ala-Pro-Val-chloromethyl ketone, was served as a positive control and it demonstrated IC50 at 80 nM.

Based on the data shown in Table 1, Rosa Freedom flower extract #2012 in Example 1 showed the highest activities in both free radical scavenging and elastase inhibition, thus was further tested against a comparative standard rose extract (which is a solvent-free extract of hydrophilic molecules).

### Example 6 Comparison of Biological Activities of Selected Rosa Freedom Flower Extract #2012 Versus a Comparative Standard Extract

Rosa Freedom flower extract #2012, which was obtained from the method of Example 1 and showed the highest biological activities in Example 5, was tested against a comparative standard rose extract and summarized in Table 2.

As shown in Table 2, both DPPH free radical scavenging and elastase inhibition activities were expressed as IC50. The lower IC50 value indicates the more desired activities, thus positive antioxidant, anti-inflammatory and antiaging benefits. Rosa Freedom flower extract #2012 demonstrated IC50 at 0.16% for DPPH free radical scavenging, which was about 10-fold higher activity than a comparative standard rose extract (with IC50 at 1.60%. In addition, Rosa Freedom flower extract #2012 showed IC50 at 0.00002% for elastase inhibition, which was about 2,000-fold higher activity than a comparative standard rose extract with IC50 at 0.04%.

The above results indicate that the rose flower extracts produced using sustainable methods of Examples 1 to 4 have demonstrated superior biological activities to a comparative standard rose extract.

### Example 7 Physico-Chemical Methods for Evaluating Rose Flower Extracts

Various methods were used to evaluate the physico-chemical and microbiological characteristics of rose flower extracts, and bioactive compositions produced according to embodiments of the process of the present invention. The test methods are described in Table 3, below.

### Example 8 Physico-Chemical Characteristics of Rose Flower Extracts

Physico-chemical and microbiological characteristics were evaluated in Rosa Freedom flower extracts in Examples 1 to 4. The test results are described in Table 4 below.

### Example 9 Preparation of Bioactive Serum Fraction and Extract Derived from Rosa “Jardin de Granville”

Rosa “Jardin de Granville” flowers and 4-5 cm of stem were harvested from rose plants. The harvesting was conducted in such a manner to avoid chopping or crushing of the collected biomass to avoid disruption of the flowers cell structure. Viability of the collected plants was tested utilizing an OS5p multi-mode chlorophyll fluorometer (Opti-Sciences Inc, Hudson, NH, USA).

The fresh live flowers including the petals, pistil and stamen were removed from the stem including the sepal and receptacle and packaged into 4-Liter plastic storage bags and placed in 4° C. storage until the harvest was complete.

Upon completion of the harvest, the rose petals were spray rinsed just prior to processing with 10° C. to 15° C. water for 0.1 to 0.3 minutes at a rate of 5 to 6 liters per minute. Excess water was removed from the rinsed flowers by allowing to drain for at least 1 minute. The rinsed flowers then underwent maceration, pressing and separation utilizing a mechanical screw press (Model CP-6 Vincent Corporation, FL) to extract the liquid intercellular colloidal dispersion (ICD) content from the fiber enriched material (“Fraction A”). The yield of Fraction A was between 40% and 55% (w/w) and the ICD contained from 6% to 12% dry matter. The ICD was placed at −20° C. storage.

The frozen ICD was then removed from storage and gently thawed.

“Treatment A” was achieved by destabilization treatment of the ICD with electromagnetic waves produced from magnetrons operating at a frequency of between 2.45 and 5.8 GHz. The parameters of the destabilization treatment were set to achieve the decrease in value of real component of low-frequency dielectric constant (ε₀′) by about 20 Farads per meter (F/m) compared to its value prior to treatment. This treatment degrades the stability of the ICD n causing agglomeration and/or aggregation of particles (i.e., organelles, organelle fragments, residual fibrous material) into assemblies which are sufficiently large and stable to enable and/or improve mechanical separation.

Mechanical separation of the ICD after “Treatment A” is achieved by centrifugation to produce “Supernatant A” and “Fraction B”.

“Supernatant A” has turbidity below about 100 NTU. “Fraction B” contains about 15.0% to 25.0% dry matter.

“Treatment B” is achieved by adjustment of pH level in “Supernatant A” by titration with, for example, alkali to obtain a pH greater than 6 up to 9.0, preferably a pH ranging from 6.5 to 7.5 or from 8.0 to 9.0, more preferably from 6.5 to 7.5, even more preferably to obtain a pH of 7.0.

Mechanical separation of “Supernatant A” after “Treatment B” is achieved by centrifugation to produce “Supernatant B” and “Fraction C”.

“Fraction C” contains about 10.0% to 20.0% dry matter.

“Treatment C” is achieved by adjustment of pH level in “Supernatant B” by titration with, for example, acid to obtain a pH lower than 5, preferably a pH ranging from 3.5 to 4.5, for example to obtain a pH of 4.0.

Mechanical separation of “Supernatant B” after “Treatment C” is achieved by centrifugation to produce the Serum Fraction of fresh Rosa de Granville petals (Unpreserved Extract) and “Fraction D”.

Serum Fraction of fresh Rosa “Jardin de Granville” petals contains from 6.0% to 10.0% dry matter.

Serum Fraction of fresh Rosa “Jardin de Granville” petals is mixed with at least one preservative or at least one stabilizer to yield a finished ingredient or combination thereof to yield the fresh Rosa “Jardin de Granville” petal extract or “Serum Rosa “Jardin de Granville”. Particularly suitable stabilizing agents can include, without limitation, a preservative, a stabilizer and/or mixtures thereof. Suitable preservatives and stabilizers for use in the present invention include, but are not limited to potassium sorbate, sodium benzoate, sodium metabisulfite, glycerin, propylene glycol, di-propylene glycol, butylene glycol, pentylene glycol, hexylene glycol, and caprylyl glycol. In a particular embodiment stabilizing agents can include at least one preservative, at least one stabilizer, at least one antioxidant or mixtures thereof.

Comparison of Biological Activities of Rose Flower Extract Derived from Rosa “Jardin De Granville” Versus a Comparative Standard Extract

Rose flower extract Derived from Rosa “Jardin de Granville”, which was obtained from the method of Example 9 was tested in both DPPH free radical scavenging assay and elastase inhibition assay and showed superior biological activities when compared to a standard rose extract.

In summary, the data presented in this disclosure indicate that the rose flower extracts produced using sustainable methods of preparation as disclosed herein have demonstrated superior biological activities to a comparative standard extract. It has been proved for potency on antioxidant, skin comfort (anti-inflammation), and dermal matrix strengthening for anti-aging benefits.

What is claimed is:

1. A method for obtaining a bioactive serum fraction derived from fresh rose flowers (Rosa spp.) comprising the steps of cleaning, macerating, pressing and mechanical separation of fresh rose flowers to obtain an intracellular colloidal dispersion (ICD) and a fiber enriched material (fraction A); treatment A and mechanical separation of the intracellular colloidal dispersion to obtain supernatant A (cytoplasm/cytosol fraction) and a membrane fraction; (fraction B); treatment B and mechanical separation of the supernatant A to obtain supernatant B (cytosol fraction) and fraction C (cytoplasm fraction); and treatment C and mechanical separation of the supernatant of supernatant B to yield the serum fraction and a fraction D (precipitate), wherein no exogenous solvent or liquid is added prior or during separating steps.

2. The method according to claim 1, wherein the rose flowers are chosen from rose belonging to any species of roses (wild roses), Old Garden Roses and Modern Roses, preferably from Modern Rose including hybrids, varieties or cultivars, more preferably from hybrid tea rose variety and even more preferably from the hybrid tea rose Rosa Freedom.

3. The method according to claim 1, wherein the rose flowers include the petals, pistil and stamen which are removed from the rose stems including the sepal and receptacle.

4. The method according to claim 1, wherein after being collected, the rose flowers are spray rinsed just prior to processing with 10° C. to 15° C. water for 0.1 to 0.3 minutes at a rate of 5 to 6 liters per minute, the excess water is removed from the rinsed flowers by allowing to drain for at least 1 minute and the rinsed flowers then undergo maceration, pressing and separation utilizing a mechanical screw press to extract the liquid intercellular colloidal dispersion (ICD) content from the fiber enriched material (fraction A).

5. The method according to claim 1 wherein the said treatment A is preceded by decrease of pH level in ICD by titration to obtain a pH lower than 4, preferably a pH ranging from 3 to 4, and more preferably to obtain a pH of 3.5, or wherein the said treatment A is preceded by a filtration.

6. The method according claim 1 wherein the said treatment A is achieved by treatment of the ICD with electromagnetic waves produced from magnetrons operating at a frequency of between 2.45 and 5.8 GHz.

7. The method according to claim 1, wherein the said treatment A is achieved by treatment of the ICD with electromagnetic waves produced from magnetrons operating at a frequency of 2.45 GHz.

8. The method according claim 1, wherein the said treatment B is achieved by increasing pH level in supernatant A to obtain a pH ranging from 6.0 to 9.0.

9. The method according to claim 1, wherein the said treatment B is achieved by increasing pH level in supernatant A to obtain a pH ranging from 6.5 to 7.5, more preferably from 6.5 to 7.5, and even more preferably to obtain a pH of 7.0.

10. The method according to claim 1, wherein the said treatment B is achieved by increasing pH level in supernatant A to obtain a pH ranging from 8.5 to 9.0, and more preferably to obtain a pH of 9.0.

11. The method according claim 1, wherein destabilization treatment C is achieved by decrease of pH level in supernatant B to obtain a pH lower than 5, preferably a pH ranging from 3.5 to 4.5, even more preferably to obtain a pH of 4.0.

12. The method according claim 1 further comprising the step of adding preservatives and/or stabilizers to yield a rose flower extract.

13. The method according to claim 1 comprising the step of adding at least one preservative chosen from potassium sorbate and sodium benzoate and/or at least one stabilizer chosen from sodium phytate as chelating agent, ascorbic acid as antioxidant, and pentylene glycol as preservative efficacy booster or a combination thereof to yield a rose flower extract.

14. A bioactive serum fraction derived from fresh rose flowers (Rosa spp.) obtainable by the method according claim 1, with the proviso that the Rosa “Jardin de Granville” is excluded.

15. The bioactive serum fraction according to claim 14, wherein said serum fraction has a dry matter ranging from 5.0% to 10.0%.

16. A bioactive extract derived from fresh rose flowers (Rosa spp.) obtainable by the method according to claim 12, with the proviso that the Rosa “Jardin de Granville” is excluded.

17. A composition comprising an effective amount of a bioactive extract derived from fresh rose flowers (Rosa spp.) obtainable by the method according to claim 12 and a physiologically acceptable medium, with the proviso that the Rosa “Jardin de Granville” is excluded.

18. The composition according to claim 17 wherein the composition is intended for topical application and is in a form selected from the group consisting of an aqueous, hydro-alcoholic or oily solution; and oil-in-water emulsion, a water-in-oil emulsion or multiple emulsions; a suspension or a powder.

19. The composition according to claim 17 wherein the composition is intended for skin care topical application in the form of a leave-on product selected from the group consisting of a cream, a dressing, a gel, a lotion, an ointment, a liquid, a spray applicator, and combinations thereof, or a wash-off product selected from the group consisting of hand dishwashing detergent, liquid hand soap, bar soap, body wash, shampoo, general purpose cleanser, and combinations thereof.

20. The composition according to claim 17 for use in skin care topical application.

21. The composition according to claim 17 for use in functional food or functional beverages.

22. A method of cosmetic treatment for improving skin appearance associated with antioxidant and antiaging properties of the bioactive serum fraction and the bioactive extract derived from fresh rose flowers (Rosa spp.), comprising applying to the area to be treated a composition according to claim 17.
